(19) United States
(12) Patent Application Publication
(10) Pub. No.: US 2022/0354469 A1
(43) Pub. Date: Nov. 10, 2022

(54) BONE BIOPSY SYSTEM AND METHOD

(71) Applicant: University of Kentucky Research Foundation, Lexington, KY (US)

(72) Inventors: Madhumathi Rao, Lexington, KY (US); Clay Larkin, Lexington, KY (US); Florence Lima, Lexington, KY (US)

(21) Appl. No.: 17/622,408

(22) PCT Filed: Jul. 2, 2020
(86) PCT No.: PCT/US20/40604

§ 371 Date: Dec. 23, 2021

Related U.S. Application Data
(60) Provisional application No. 62/869,759, filed on Jul. 2, 2019.
(60) Provisional application No. 62/991,803, filed on Mar. 19, 2020.

(51) Int. Cl. A61B 10/02

(52) U.S. Cl. CPC A61B 10/025; A61B 10/0266

(57) ABSTRACT

A bone biopsy system includes a stylet, a trephine needle, a drive connector and a push rod. The trephine needle includes a lumen and a cutting edge. The stylet is received in the lumen to set the trephine needle in position for procuring a bone core sample. The stylet is then removed and the trephine needle is driven into the bone to collect a bone core sample in the lumen.

### RELATED APPLICATION

This application claims priority to U.S. Provisional Patent Application Ser. No. 62/869,759 filed on Jul. 2, 2019 and U.S. Provisional Patent Application Ser. No. 62/991,803 filed on Mar. 19, 2020 which is hereby incorporated by reference in its entirety.

### TECHNICAL FIELD

This document relates generally to a bone biopsy system as well as to a related method of procuring a bone core sample from a bone of a patient.

### BACKGROUND

A bone biopsy is a medical procedure where a core of bone is obtained from the iliac crest of a patient for diagnostic purposes. It is the gold standard for diagnosing and guiding treatment for a wide range of metabolic bone disorders.

There is currently no dedicated bone biopsy needle on the market. The centers that perform this procedure use and adapt bone marrow biopsy needles or power-driven handheld systems generally used for bone access by orthopedic surgeons.

This document relates to a novel (a) bone biopsy system and (b) related method of procuring a bone core sample from a bone of a patient all dedicated to the bone biopsy procedure. The design for the needle is specific to the bone biopsy procedure and minimizes tissue injury and complications to the patient. In addition, the bone biopsy system, incorporating that bone biopsy needle, and the related method of procuring a bone core sample from the bone of a patient address ease of use for the operator.

### SUMMARY

In accordance with the purposes and benefits described herein, a novel bone biopsy system comprises (a) a stylet, including an elongated body having a pointed tip at a distal end and a first drive connector at a proximal end, and (b) a trephine needle, including an elongated shaft having a lumen, a cutting edge at a first end and a tool connector at a second end wherein the stylet is received in the lumen when in an operating position. In one or more of the many possible embodiments of the bone biopsy system, the pointed tip of the stylet projects from the cutting edge of the trephine needle when the stylet is in the operating position.

In one or more of the many possible embodiments of the bone biopsy system, the stylet includes a needle connector that releasable engages with the tool connector when the stylet is in the operating position.

In one or more of the many possible embodiments of the bone biopsy system, the lumen is tapered toward the cutting edge.

In one or more of the many possible embodiments of the bone biopsy system, the cutting edge is located on an outer surface of the trephine needle.

In one or more of the many possible embodiments of the bone biopsy system, the cutting edge is a low-helix. That low-helix may have a rake angle of between 34 and 36 degrees. The cutting edge may taper in both directions from a midline. Further, the cutting edge may be reduced to an extension of about 0.05 mm at a cutting terminal and 0.00 mm at a luminal end for a flush fit.

In one or more of the many possible embodiments of the bone biopsy system, the lumen taper is 0.75 mm in total and terminates within 13.2 mm of the luminal end.

In one or more of the many possible embodiments of the bone biopsy system, the bone biopsy system further includes a second drive connector having a needle connector that releasably engages with the tool connector when the stylet is removed from the lumen of the trephine needle.

In one or more of the many possible embodiments of the bone biopsy system, the bone biopsy system further includes a push rod adapted for forcing a bone core sample from the lumen of the trephine needle.

In one or more of the many possible embodiments of the bone biopsy system, the bone biopsy system further includes a powered driver releasably connected to (a) the first drive connector of the stylet connected to the trephine needle when seating the cutting edge of the trephine needle in a bone of a patient and (b) the second drive connector connected to the trephine needle when procuring the bone core sample in the lumen.

In accordance with yet another aspect, a bone biopsy system is provided. That bone biopsy system comprises (a) a stylet, (b) a trephine needle having a lumen and a cutting edge and (c) a drive connector. The stylet is connected to the trephine needle to seat the cutting edge into a bone of a patient and, subsequently, the stylet is removed from the trephine needle and the drive connector is connected to the trephine needle to procure a bone core sample in the lumen.

In one or more of the many possible embodiments of the bone biopsy system, the bone biopsy system further includes a powered driver releasably connected to the stylet when seating the cutting edge and releasably connected to the drive connector when procuring the bone core sample.

In one or more of the many possible embodiments of the bone biopsy system, the bone biopsy system further includes a push rod adapted to force the bone core sample from the lumen.

In accordance with still another aspect, a method is provided for procuring a bone core sample from a bone of a patient. That method comprises the steps of: (a) seating a cutting edge of a trephine needle in the bone by first engaging a stylet in the bone and thereby holding the trephine needle in a seating position, (b) removing the stylet from the trephine needle (c) connecting a drive connector to the trephine needle and (d) driving the trephine needle into the bone and securing the bone core sample within a lumen of the trephine needle.

In one or more embodiments, the method further includes the step of connecting a powered driver to the stylet before seating the cutting edge in the bone.

In one or more embodiments, the method further includes the step of connecting the powered driver to the drive connector before driving the trephine needle into the bone.

In one or more embodiments, the method further includes the steps of removing the trephine needle from the bone, removing the drive connector from the trephine needle and inserting a push rod into the lumen of the trephine needle to force the bone core sample from the lumen.

In one or more embodiments, the method further includes the steps of inserting the stylet into the lumen of the trephine needle and connecting the stylet to the trephine needle before seating the cutting edge.

In the following description, there are shown and described several embodiments of the novel (a) bone biopsy system and (b) related method of procuring a bone core sample from a bone of a patient. As it should be realized, the bone biopsy system and related method are capable of other, different embodiments and their several details are capable of modification in various, obvious aspects all without departing from the bone biopsy system and method as set forth and described in the following claims. Accordingly, the drawings and descriptions should be regarded as illustrative in nature and not as restrictive.

### BRIEF DESCRIPTION OF THE DRAWING FIGURES

The accompanying drawing figures incorporated herein and forming a part of the specification, illustrate several aspects of the novel bone biopsy system and related method of procuring a bone core sample from a bone of a patient and together with the description serve to explain certain principles thereof.

FIG. 1 is a side elevational view of the bone biopsy system in a disassembled state, including, from left to right, the powered driver, the stylet, the drive connector, the trephine needle and the push rod.

FIG. 2 is a detailed longitudinal cross-sectional view of the cutting edge of the trephine needle.

FIG. 3 is a detailed first end/cutting edge view of the trephine needle.

FIG. 4a is side elevational view illustrating the biopsy needle with the stylet inserted into the lumen of the trephine needle and the pointed tip of the stylet projecting from the first end of the trephine needle.

FIG. 4b is a detailed longitudinal cross-sectional view of a portion of the structure illustrated in FIG. 4a.

FIG. 5 is a side elevational view of the bone biopsy needle with the stylet removed from the lumen of the trephine needle and the drive connector connected to the second end of the trephine needle.

FIG. 6a is a side elevational view illustrating the bone biopsy needle with the push rod inserted into the lumen of the trephine needle and forcing a bone core sample from the second end of the trephine needle.

FIG. 6b is a detailed longitudinal cross-sectional view of a portion of the trephine needle showing the push rod being pushed through the lumen of the trephine needle in order to force a bone core sample from the lumen.

FIG. 7 is a schematic illustration of the step of seating a cutting edge of the trephine needle in the bone by engaging the pointed tip of the stylet into the bone.

FIG. 8 is a schematic illustration of the step of driving the trephine needle into the bone to secure the bone core sample in the lumen of the trephine needle.

### DETAILED DESCRIPTION

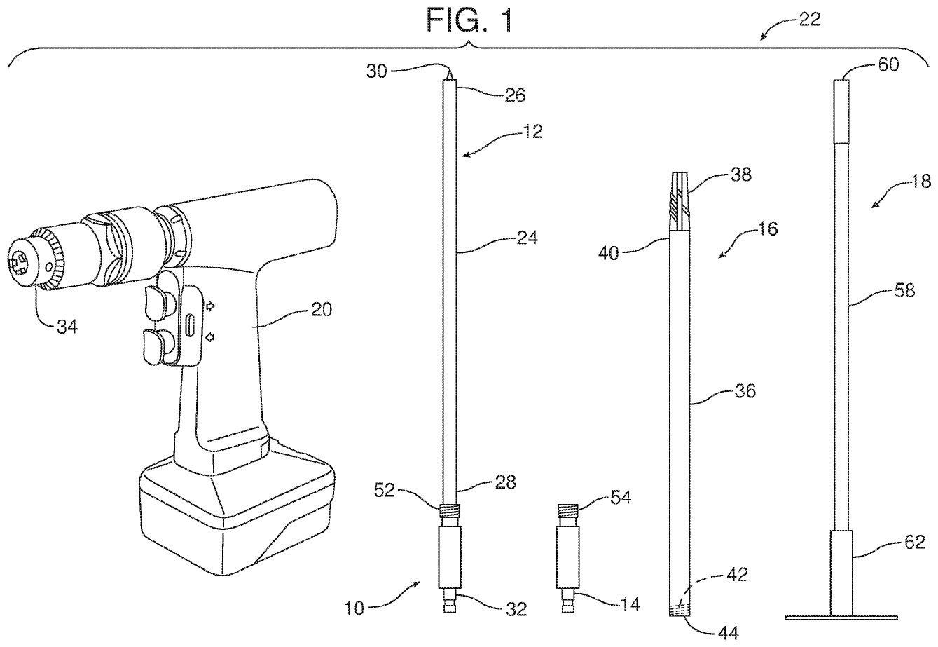

Reference is now made to FIG. 1 which illustrates the various components of the novel bone biopsy needle assembly 10 in a disassembled state or condition. As shown, the bone biopsy needle assembly 10 generally includes a stylet 12, a drive connector 14, a trephine needle 16 and the push rod 18. FIG. 1 also shows a powered driver 20. Together, the bone biopsy needle assembly 10 and the powered driver 20 comprise the novel bone biopsy system 22.

The stylet 12 includes an elongated body 24 having a distal end 26 and a proximal end 28. The stylet 12 includes a pointed tip 30 at the distal end 26 and a drive connector 32 at the proximal end 28. The drive connector 32 may be of any appropriate design for mating with powered driver 20 and, more particularly with the chuck 34 of the powered driver. In one example, the drive connector 32 may include a Trinkle connection for mating with a powered driver 20 such as a Stryker CD4 POWERream driver. The stylet 12 may be made from surgical stainless-steel alloy, titanium alloy or other appropriate material. Recommended driver specifications include high torque and moderate (approximately 1,400-1,600) rpm.

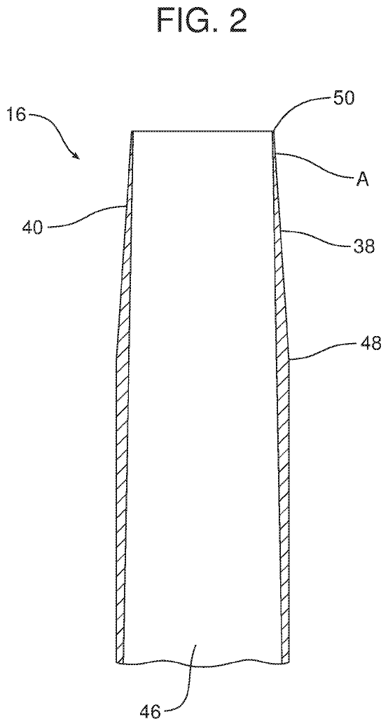

The trephine needle 16 includes an elongated shaft 36, a cutting edge 38 at a first end 40 of the elongated shaft and a tool connector 42 at a second end 44 of the elongated shaft. A lumen 46 extends through the entire longitudinal length of the elongated shaft 36. That lumen 46 may taper toward the cutting edge 38 provided at the first or luminal end 40 of the elongated shaft 36 (see also FIG. 2). The lumen taper may be 0.75 mm in total and terminate within 13.2 mm of the first or luminal end 40.

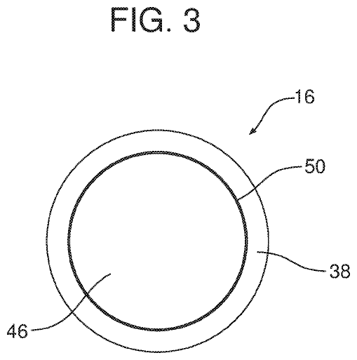

More specifically, as best illustrated in FIGS. 2 and 3, the cutting edge 38 is located on the outer surface of the trephine needle 16. The cutting edge 38 may be of low-helix design and have a rake angle A of between 34-36 degrees and, more specifically about 34.7 degrees. In the illustrated embodiment, the cutting edge 38 tapers in both directions from the midline 48 and may have a thickness of about 0.05 mm at a cutting terminal 50 and a thickness of about 0.2 mm at the opposite end. The midline 48 thickness is about 0.375 mm. The trephine needle 16, including the cutting edge 38 may be made from titanium alloy, tantalum alloy, platinum alloy, stainless-steel alloy or other appropriate material.

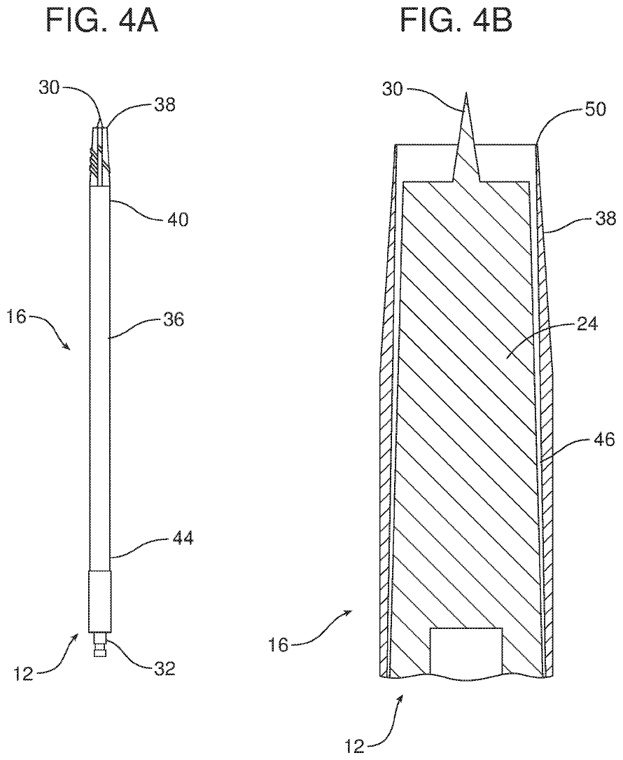

Reference is now made to FIGS. 4a and 4b which illustrate the stylet 12 in the operating position wherein the stylet is received in the lumen 46 of the trephine needle 16. More particularly, when the stylet 12 is properly seated in the lumen 46 of the trephine needle 16, the pointed tip 30 projects from the first end 40 of the trephine needle, past the cutting edge 38.

As illustrated in FIG. 1, the stylet 12 also includes a needle connector 52 that releasably engages the tool connector 42 of the trephine needle 16 when the stylet is in the operating position. More specifically, in the illustrated embodiment the needle connector 52 and the tool connector 42 comprise mating threads. Thus, one properly seats the stylet 12 in the trephine needle 16 by engaging the inner threads of the needle connector 52 with the outer threads of the tool connector 42 and twisting to draw the stylet fully into the lumen 46 of the trephine needle 16.

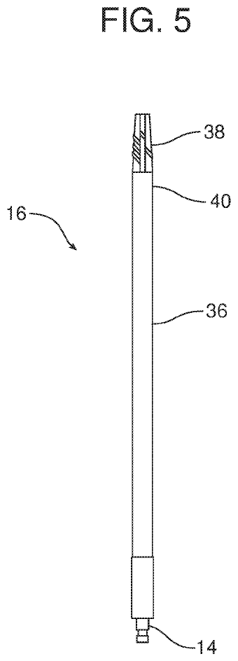

As illustrated in FIGS. 1 and 5, the independent drive connector 14 also includes a needle connector 54 that releasably engages with the tool connector 42 of the trephine needle 16 when the stylet 12 is removed from the trephine needle and the drive connector 14 is connected to the trephine needle. Once again the needle connector 54, like the needle connector 52 may comprise threads that mate with the threads of the tool connector 42.

As illustrated in FIG. 1, the push rod 18 includes an elongated body 58 having a plunger tip 60 at one end and a stop 62 at the other end.

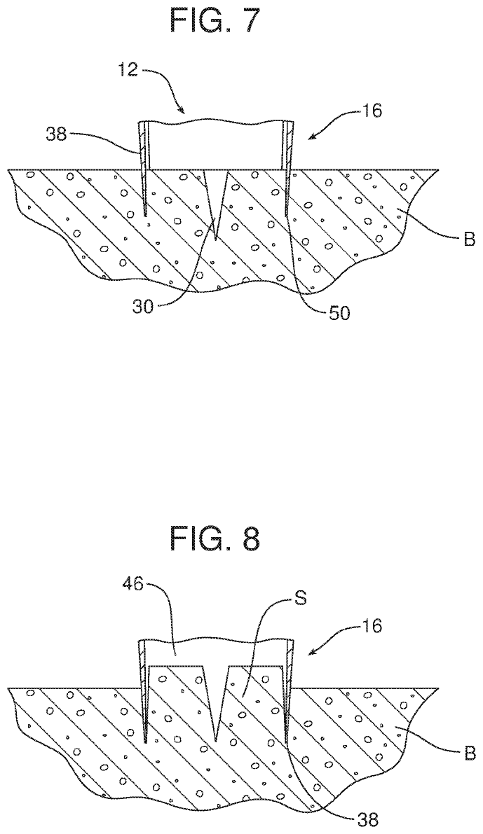

The bone biopsy needle 10 and the bone biopsy system 22 are useful in a method of procuring a bone core sample from a bone B of a patient. That method includes the step of seating the cutting edge 38 of the trephine needle 16 in the bone B by first engaging the stylet 12 in the bone and thereby holding the trephine needle in a seating position (see FIG. 7). Toward this end, the stylet 12 is placed in the operating position within the lumen 46 of the trephine needle 16 as described above and illustrated in FIGS. 4a and 4b. The adjustable chuck 34 of the powered driver 20 is then connected to the drive connector 32 on the stylet 12.

As shown in FIGS. 4a, 4b and 7, the pointed tip 30 of the stylet 12 projects beyond the cutting edge 38 of the trephine needle 16. Thus, as the powered driver 20 begins to turn the bone biopsy needle 10 at the bone B, the pointed tip 30 engages and drills into the bone B before the cutting edge engages the bone. This sets the position of the bone biopsy needle 10 with respect to the bone B, thereby seating and holding the cutting edge 38 in a proper seating position for procuring a bone core sample.

Once the bone biopsy needle 16 is properly seated, the needle connector 52 on the stylet 12 is disconnected from the tool connector 42 on the trephine needle 16. The stylet 12 is then withdrawn from second end 44 of the trephine needle 16 while the cutting edge 38 of the trephine needle is seated in the bone B.

After removing the stylet 12 from the trephine needle 16, the drive connector 14 is connected to the trephine needle by engaging the needle connector 54 of the drive connector 14 with the tool connector 42 on the trephine needle. Next, the powered driver 20 is connected to the drive connector 14 by engaging the chuck 34 with the drive connector 14. This is all done while maintaining the trephine needle 16 in the seating position with the bone B.

Next is the step of driving the trephine needle 16 into the bone B with the powered driver 20 and securing the bone core sample S within the lumen 46 of the trephine needle 16 (see FIG. 8). The tapering of the lumen 46 aids in allowing the bone core sample S to be drawn upward into the lumen 46 during the driving operation.

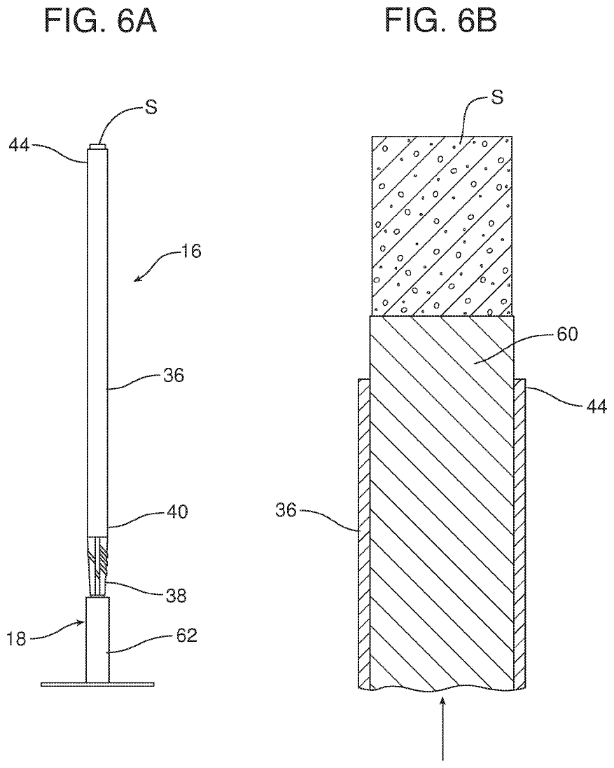

Once the bone core sample S has been procured from the bone B, the method includes the step of removing the trephine needle 16 from the bone B. This is followed by the step of removing the drive connector 14 from the trephine needle 16 by disengaging the needle connector 54 on the drive connector 14 from the tool connector 42 of the trephine needle 16. This is then followed by the step of inserting the push rod 18 into the lumen 46 of the trephine needle 16 to force the bone core sample S from the lumen. More particularly, as best illustrated in FIGS. 6a and 6b, the plunger tip 60 of the push rod 18 is inserted into the first end 40 of the trephine needle 16 and pushed through the lumen 46 toward the second end 44: that is from the cutting edge 38 toward the tool connector 42 until the bone core sample S is ejected from the lumen of the trephine needle. The stop 62 prevents the push rod 18 from passing through the lumen 46 and provides a gripping point for manipulating the push rod 18.

The bone core sample S is then collected for analysis.

This disclosure may be considered to relate to the following items.

1. A bone biopsy system, comprising:

a stylet including an elongated body having a pointed tip at a distal end and a first drive connector at a proximal end; and

a trephine needle including an elongated shaft having a lumen, a cutting edge at a first end and a tool connector at a second end wherein said stylet is received in said lumen when in an operating position.

2. The bone biopsy system of item 1, wherein the pointed tip projects from the cutting edge when the stylet is in the operating position.

3. The bone biopsy system of item 1 or item 2, wherein said stylet includes a needle connector that releasable engages with the tool connector when the stylet is in the operating position.

4. The bone biopsy system of any of items 1-3, wherein said lumen is tapered toward the cutting edge.

5. The bone biopsy system of any of items 1-4, wherein the cutting edge is located on an outer surface of the trephine needle.

6. The bone biopsy system of any of items 1-5, wherein the cutting edge is a low-helix.

7. The bone biopsy system of item 6, wherein the low-helix has a rake angle of between 34 and 36 degrees.

8. The bone biopsy system of any of items 1-7, wherein the cutting edge tapers in both directions from a midline.

9. The bone biopsy system of any of items 1-8, wherein the cutting edge is reduced to an extension of about 0.05 mm at a cutting terminal and 0.00 mm at the first end for a flush fit.

10. The bone biopsy system of any of items 1-9, wherein the lumen taper is 0.75 mm in total and terminates within 13.2 mm of the first end.

11. The bone biopsy system of any of items 1-10, further including a second drive connector having a needle connector that releasable engages with the tool connector when the stylet is removed from the lumen of the trephine needle.

12. The bone biopsy system of any of items 1-11, further including a push rod adapted for forcing a bone core sample from the lumen of the trephine needle.

13. The bone biopsy system of any of items 1-12, further including a powered driver releasably connected to (a) the first drive connector of the stylet connected to the trephine needle when seating the cutting edge of the trephine needle in a bone of a patient and (b) the second drive connector connected to the trephine needle when procuring the bone core sample in the lumen.

14. A bone biopsy system, comprising:

a stylet;

a trephine needle having a lumen and a cutting edge; and

a drive connector wherein the stylet is connected to the trephine needle to seat the cutting edge into a bone of a patient and the stylet is removed from the trephine needle and the drive connector is connected to the trephine needle to procure a bone core sample in the lumen.

15. The bone biopsy system of item 14, further including a powered driver releasably connected to the stylet when seating the cutting edge and releasably connected to the drive connector when procuring the bone core sample.

16. The bone biopsy system of any of items 14-15, further including a push rod adapted to force the bone core sample from the lumen.

17. A method of procuring a bone core sample from a bone, comprising:

seating a cutting edge of a trephine needle in the bone by first engaging a stylet in the bone and thereby holding the trephine needle in a seating position;

removing the stylet from the trephine needle;

connecting a drive connector to the trephine needle; and

driving the trephine needle into the bone and securing the bone core sample within a lumen of the trephine needle.

18. The method of item 17, including connecting a powered driver to the stylet before seating the cutting edge in the bone.

19. The method of any of items 17-18, including connecting the powered driver to the drive connector before driving the trephine needle into the bone.

20. The method of any of items 17-19, including removing the trephine needle from the bone, removing the drive connector from the trephine needle and inserting a push rod into the lumen of the trephine needle to force the bone core sample from the lumen.

21. The method of any of items 17-20, including inserting the stylet into the lumen of the trephine needle and connecting the stylet to the trephine needle before seating the cutting edge.

Numerous benefits and advantages are provided by the bone biopsy system 22 and related method of procuring a bone core sample. The powered driver 20 reduces the time required to complete the method/procedure and reduces the physical strain on the physician performing the procedure. The powered driver 20 also gives more consistent and uniform results.

The stylet 12 functions as an introducer and anchors the direction of the drilling until the cutting edge 38 of the trephine needle 16 engages into the cortex of the bone B. The stylet 12 effectively prevents the cutting edge 38 from slipping over or “walking” along the surface of the bone B as the needle 16 rotates.

In a typical application, the stylet 12 would be removed from the trephine needle 16 as soon as the cutting edge 38 is properly seated into the bone (approximately 2 mm). Thus, correct positioning of the trephine needle 16 to obtain the desired bone core sample is ensured.

The cutting edge 38 allows the bone sample to be collected without interfering with the microarchitecture of the sample. The rake angle of the trephine needle 16 is adapted to specifically reduce heat gain and bone micro fracturing due to friction and resistance during the obtaining of the sample. The cutting edge 38 is also angled to reduce innate resistance that would exist if the cutting edge 38 was designed to enter the bone perpendicularly.

The tapered lumen 46 aides both insertion of the trephine needle 16 into the bone B as well as retrieval of the bone core sample from the trephine needle with the push rod 18.

Each of the following terms written in singular grammatical form: “a”, “an”, and the“, as used herein, means “at least one”, or “one or more”. Use of the phrase One or more” herein does not alter this intended meaning of “a”, “an”, or “the”. Accordingly, the terms “a”, “an”, and “the”, as used herein, may also refer to, and encompass, a plurality of the stated entity or object, unless otherwise specifically defined or stated herein, or, unless the context clearly dictates otherwise. For example, the phrases: “a unit”, “a device”, “an assembly”, “a mechanism”, “a component, “an element”, and “a step or procedure”, as used herein, may also refer to, and encompass, a plurality of units, a plurality of devices, a plurality of assemblies, a plurality of mechanisms, a plurality of components, a plurality of elements, and, a plurality of steps or procedures, respectively.

Each of the following terms: “includes”, “including”, “has”, “having”, “comprises”, and “comprising”, and, their linguistic/grammatical variants, derivatives, or/and conjugates, as used herein, means “including, but not limited to”, and is to be taken as specifying the stated component(s), feature(s), characteristic(s), parameter(s), integer(s), or step(s), and does not preclude addition of one or more additional component(s), feature(s), characteristic(s), parameter(s), integer(s), step(s), or groups thereof. Each of these terms is considered equivalent in meaning to the phrase “consisting essentially of.” Each of the phrases “consisting of and “consists of, as used herein, means “including and limited to”. The phrase “consisting essentially of” means that the stated entity or item (system, system unit, system sub-unit device, assembly, sub-assembly, mechanism, structure, component element or, peripheral equipment utility, accessory, or material, method or process, step or procedure, sub-step or sub-procedure), which is an entirety or part of an exemplary embodiment of the disclosed invention, or/and which is used for implementing an exemplary embodiment of the disclosed invention, may include at least one additional feature or characteristic” being a system unit system sub-unit device, assembly, sub-assembly, mechanism, structure, component or element or, peripheral equipment utility, accessory, or material, step or procedure, sub-step or sub-procedure), but only if each such additional feature or characteristic” does not materially alter the basic novel and inventive characteristics or special technical features, of the claimed item.

The term “method”, as used herein, refers to steps, procedures, manners, means, or/and techniques, for accomplishing a given task including, but not limited to, those steps, procedures, manners, means, or/and techniques, either known to, or readily developed from known steps, procedures, manners, means, or/and techniques, by practitioners in the relevant field(s) of the disclosed invention.

Terms of approximation, such as the terms about, substantially, approximately, etc., as used herein, refers to ±10% of the stated numerical value. Use of the terms parallel or perpendicular is meant to mean approximately meeting this condition, unless otherwise specified.

The foregoing has been presented for purposes of illustration and description. It is not intended to be exhaustive or to limit the embodiments to the precise form disclosed. Obvious modifications and variations are possible in light of the above teachings. All such modifications and variations are within the scope of the appended claims when interpreted in accordance with the breadth to which they are fairly, legally and equitably entitled.

1. A bone biopsy system, comprising:
a stylet including an elongated body having a pointed tip at a distal end and a first drive connector at a proximal end; and
a trephine needle including an elongated shaft having a lumen, a cutting edge at a first end and a tool connector at a second end wherein said stylet is received in said lumen when in an operating position.

2. The bone biopsy system of claim 1, wherein the pointed tip projects from the cutting edge when the stylet is in the operating position.

3. The bone biopsy system of claim 1, wherein said stylet includes a needle connector that releasable engages with the tool connector when the stylet is in the operating position.

4. The bone biopsy system of claim 1, wherein said lumen is tapered toward the cutting edge.

5. The bone biopsy system of claim 1, wherein the cutting edge is located on an outer surface of the trephine needle.

6. The bone biopsy system of claim 1, wherein the cutting edge is a low-helix.

7. The bone biopsy system of claim 6, wherein the low-helix has a rake angle of between 34 and 36 degrees.

8. The bone biopsy system of claim 7, wherein the cutting edge tapers in both directions from a midline.

9. The bone biopsy system of claim 8, wherein the cutting edge is reduced to an extension of about 0.05 mm at a cutting terminal and 0.00 mm at the first end for a flush fit.

10. The bone biopsy system of claim 4, wherein the lumen taper is 0.75 mm in total and terminates within 13.2 mm of the first end.

11. The bone biopsy system of claim 1, further including a second drive connector having a needle connector that releasably engages with the tool connector when the stylet is removed from the lumen of the trephine needle.

12. The bone biopsy system of claim 11, further including a push rod adapted for forcing a bone core sample from the lumen of the trephine needle.

13. The bone biopsy system of claim 12, further including a powered driver releasably connected to (a) the first drive connector of the stylet connected to the trephine needle when seating the cutting edge of the trephine needle in a bone of a patient and (b) the second drive connector connected to the trephine needle when procuring the bone core sample in the lumen.

14. A bone biopsy system, comprising:
a stylet;
a trephine needle having a lumen and a cutting edge; and
a drive connector wherein the stylet is connected to the trephine needle to seat the cutting edge into a bone of a patient and the stylet is removed from the trephine needle and the drive connector is connected to the trephine needle to procure a bone core sample in the lumen.

15. The bone biopsy system of claim 14, further including a powered driver releasably connected to the stylet when seating the cutting edge and releasably connected to the drive connector when procuring the bone core sample.

16. The bone biopsy system of claim 14, further including a push rod adapted to force the bone core sample from the lumen.

17. A method of procuring a bone core sample from a bone, comprising:
seating a cutting edge of a trephine needle in the bone by first engaging a stylet in the bone and thereby holding the trephine needle in a seating position;
removing the stylet from the trephine needle;
connecting a drive connector to the trephine needle; and
driving the trephine needle into the bone and securing the bone core sample within a lumen of the trephine needle.

18. The method of claim 17, including connecting a powered driver to the stylet before seating the cutting edge in the bone.

19. The method of claim 18, including connecting the powered driver to the drive connector before driving the trephine needle into the bone.

20. The method of claim 19, including removing the trephine needle from the bone, removing the drive connector from the trephine needle and inserting a push rod into the lumen of the trephine needle to force the bone core sample from the lumen and inserting the stylet into the lumen of the trephine needle and connecting the stylet to the trephine needle before seating the cutting edge.

21. (canceled)
